(19) United States
(12) Patent Application Publication
(10) Pub. No.: US 2018/0289612 A1
(43) Pub. Date: Oct. 11, 2018

(54) COSMECEUTICAL

(71) Applicant: Keprea, LLC, New York, NY (US)

(72) Inventors: Stephen Squinto, New York, NY (US); Denise LePera, New York, NY (US)

(21) Appl. No.: 15/574,706

(22) PCT Filed: May 17, 2016
(86) PCT No.: PCT/US2016/032892

§ 371 Date: Nov. 22, 2017

Related U.S. Application Data
(60) Provisional application No. 62/166,898, filed on May 27, 2015.

(51) Int. Cl. A61K 8/9789; A61Q 19/00; A61Q 19/08

(52) U.S. Cl. CPC A61K 8/9789; A61Q 19/08; A61Q 19/00

(57) ABSTRACT

The invention relates to a composition for skin improvement or repair, comprising an extract of Boswellia frereana; a method of producing said composition; and a method of skin improvement or repair comprising use of said composition.

This application is the national stage of international patent application no. PCT/US2016/032892 filed on May 17, 2016 which in turn claims priority from U.S. Patent Application No. 62/166,898 filed on May 27, 2015, the disclosures of which are incorporated herein by reference in their entirety.

### TECHNICAL FIELD

The invention relates to a composition for skin improvement or repair, comprising an extract of Boswellia frereana; a method of producing said composition; and a method of skin improvement or repair comprising use of said composition.

### BACKGROUND

Many recent cosmetic products and techniques are based on advanced scientific research that includes the use of biotechnologically derived ingredients, nutritional regimes, stem-cell-based products and therapies to regenerate ageing tissues, or use cell and tissue engineering for cosmetic purposes. ‘Cosmeceutical’ is a term that has arisen from this concept and typically refers to the combination of cosmetics with biologically active ingredients to promote medical or drug-like benefits.

Through improved understanding of the structure of the skin and its underlying repair and maintenance processes, researchers are increasingly able to intervene to reduce the effects of premature ageing or improve healing processes. The cosmetics and pharmaceutical industries have also expended considerable effort to understand the ageing processes of the skin and to devise countermeasures.

The dermal layer of the skin is composed of multiple cell types immersed in extracellular matrix containing collagen, elastin and polysaccharides. Skin naturally becomes thinner with age, as it loses its underlying fat layer. Additionally, the amount of oil the skin produces also decreases with age leading to dryness. As we age the elastic fibers in skin, like collagen and elastin, begin to degrade and are not renewed.

In this respect, it is thought that skin aging and loss of elasticity are related mainly to a decrease in collagen and elastin fibers. In the extracellular matrix forming the dermis, the quantity and quality of extracellular collagen is determined by the balance between collagen synthesis and degradation. It has been suggested that low grade inflammation leads to a chronic asymptomatic pro-inflammatory state where tissue damaging mechanisms lead to aging of the skin, resulting in development of wrinkles, loss of collagen, elastin fibers and elasticity, and accumulation of subtle tissue damage.

Boswellia is a genus of trees in the order Sapindales. The sap from the trees is commonly known as frankincense, which is a fragrant resin used in perfumery and aromatherapy. There are four main species of Boswellia which produce true frankincense. B. sacra (synonyms B. carteri and B. bhaw-dajiana), B. frereana, B. papyrifera, and B. serrata.

In addition, frankincense has many pharmacological uses, particularly as an anti-inflammatory. In Ayurvedic medicine, frankincense from Boswellia serrata, commonly referred to in India as “dhoop,” has been used for hundreds of years for treating arthritis, healing wounds, strengthening the female hormone system and purifying the air.

Preparations from the oleo resin of the Boswellia genus (other than B. frereana), have been identified as potent anti-inflammatory, anti-arthritic and anti-analgesic agents. Traditionally, Boswellia serrata species has been used extensively in treating conditions which are either initiated by, or maintained by, inflammatory events. The main pharmacologically active ingredients of B. serrata are α- and β-boswellic acids which are well known for their anti-inflammatory properties.

However, it has previously been shown that B. frereana does not contain boswellic acid (alpha or beta), the main pharmacologically active ingredient of B. Serrata, but nonetheless this species is effective as an anti-inflammatory and interestingly this anti-inflammatory effect operates, at least partly, via the suppression, deactivation or inhibition of matrix metalloproteinase 9 (MMP9). It was found that the key ingredient pertaining to the pharmacological effect was Epi-lupeol, a diastereisomer of the pentacyclic triterpene lupeol.

We described herein a novel cosmeceutical based upon an extract of B. frereana.

### SUMMARY

According to a first aspect of the invention there is provided a composition formulated for topical application for use in skin improvement or repair comprising an extract of Boswellia frereana, wherein said extract is present in an amount at or between 1-3% by weight of the formulation.

Reference herein to skin improvement or repair refers to any process whose purpose is to improve the functionality of the skin, overcome defects or achieve a desired particular outcome such as overall appearance or restoration. This may include, but is not limited to, an improvement in at least one of: skin texture and re-pigmentation, skin smoothing and wrinkle removal, skin firming, skin radiance, skin plumping, skin clarity, skin regeneration, skin moisture and hydration, improved skin barrier functionality, improved skin elasticity and firmness, extracellular matrix stimulation and maintenance, or the like.

Reference herein to an extract of Boswellia frereana (B. Frereana) refers to a mixture of components which are present in the oleo-resin of Boswellia frereana and which are soluble in a suitable organic solvent. The extract may be obtained by mixing the gum resin with the solvent and leaving for a suitable period of time, for example several days, ideally in a light-free environment so that the soluble components of the gum are extracted into the solvent. The solvent may then be removed to leave the extract. For the avoidance of doubt, “extract” refers to both a single component and to a mixture of components.

In a preferred embodiment of the invention, said solvent may be a polar solvent, typically an alcohol such as, but not limited to methanol, ethanol, isoproponal, methyl isopropanol and derivatives thereof or a non polar solvent such as, but not limited to, hexane. Most preferably, said solvent is methyl isopropanol.

Alternatively, the extraction may also be carried out using other extraction solvents such as ethyl acetate, diethyl ether, chloroform, methylene chloride, petroleum ether, acetone, pentane, or toluene. Other suitable solvents will be well known to those skilled in the art of plant component extraction.

In yet a further preferred embodiment of the invention, said extract is the essential oil of Boswellia frereana. Yet more preferably still, said extract does not contain Boswellic acid.

In a preferred embodiment of the invention, said extract is ideally further solubilized in an oil such as, but not limited to, jojoba.

Typically, the extract is present in an amount at or between 1-3% by weight of the formulation and more ideally still selected from the group comprising or consisting of: 1%, 2%, 3% and every 0.1% integer there between.

Reference herein to “% by weight of the formulation” means the percent concentration of the component in the formulation in a weight/weight basis. For example, 1% w/w of component extract=[(mass of component extract)/(total mass of the formulation)]×100.

Therefore, in a preferred embodiment of the invention, the extract is present in an amount between 1-3% by weight of the formulation and more ideally still selected from the group comprising or consisting of: 1%, 2%, 3% and every 0.1% integer there between and wherein the extract is the essential oil of Boswellia frereana. More preferably still, said extract does not contain Boswellic acid.

Preferably said extract comprises the pentacyclic triterpene epi-lupeol and/or lupeol or a derivative or a salt thereof. The term “derivative” includes but is not limited to ether derivatives, acid derivatives, amide derivatives, ester derivatives and the like. Suitable derivatives include ester derivatives such as benzoic, phosphate, octanoate esters etc.; acetate esters in particular epilupeol acetate.

The invention also includes the use of pharmaceutically acceptable salts of lupeol and/or epi-lupeol. As used herein, the term “pharmaceutically acceptable salt” refers to a compound formulated from a base compound which achieves substantially the same pharmaceutical effect as the base compound. Suitable pharmaceutically or veterinary acceptable salts include basic addition salts such as sodium, potassium, calcium, aluminum, zinc, magnesium and other metal salts as well as choline, diethanolamine, ethanolamine, ethyl diamine, megulmine and other well-known basic addition salts are known to those skilled in the art.

Epi-lupeol is a diastereisomer of the pentacyclic triterpene lupeol. Diastereomers are defined as stereoisomers that are not enantiomers (i.e non-superimposable mirror images of each other). Diastereomers, sometimes called diastereoisomers generally have different physical properties and different reactivities to their isomers.

Other components of the extract comprise β-amyrin, α-amyrin, α-phellandrene dimers, α-thujene and α-phellandrene and their isomers and, where appropriate, salts. More preferably the main components are epi-lupeol, β-amyrin and alpha-phellandrene dimer.

In a preferred embodiment of the invention said composition further comprises at least one carrier or excipient, or, if more than one is present, at least one of each of the carriers or excipients or a plurality of at least one carrier or at least one excipient; wherein said carrier or excipient refers to any compound that is acceptable in the sense of being compatible with the other ingredients of the formulation and not deleterious to the recipient.

Other active materials may also be present in the formulation according to invention, as may be considered appropriate or advisable for skin repair or improvement. For example, the composition may also contain a moisturiser or emollient, or the like.

The composition may be prepared by bringing into association the extract of the invention and the carrier. In general, the formulations are prepared by uniformly and intimately bringing into association the extract of the invention with liquid carriers or gel/cream carriers or finely divided solid carriers or mixtures thereof, including any combination, and then if necessary shaping the product. The invention extends to methods for preparing a composition comprising bringing an extract of the invention in conjunction or association with a pharmaceutically or veterinary acceptable carrier or excipient.

For topical application to the skin, said composition may be made up into a cream, ointment, jelly, serum, mist, face mask, lotion, solution or suspension or the like. Cream or ointment formulations that may be used for the extract are conventional formulations well known in the art, for example, as described in standard text books of pharmaceutics such as the British Pharmacopoeia.

In a preferred embodiment of he invention, said composition is formulated as a cream.

According to a further aspect of the invention there is provided a method for skin improvement or repair comprising applying an effective amount of the composition according to the invention to the surface of the skin of an individual.

In a preferred embodiment of the method, said composition is applied at least twice daily to the surface of the skin.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The Invention will now be described by way of example only with reference to the Examples below and to the following Figures wherein:

Table 1. Skin Firmness Analysis. Measurements were taken after 2, 4 and 8 weeks of product use of each formulation (1%, 2%, and 3% Boswellia frereana (BF) extract formulation and placebo) and compared with baseline measurements based on conventional Cutometer® readings. All readings were assessed at a significance of p<0.001;

Table 2. Statistical Significance of the effect of Boswellia frereana (BF) extract formulations versus Placebo upon skin firmness. Statistical comparisons were performed comparing groups A, B and C against group D through 8 weeks;

Table 3. Skin Moisture/Hydration Analysis. Measurements were taken after 2, 4 and 8 weeks of product use of each formulation (1%, 2%, and 3% Boswellia frereana (BF) extract formulation and placebo) and compared with baseline measurements based on conventional Corneometer® readings. All readings were assessed at a significance of p<0.001;

Table 4. Statistical Significance of the effect of Boswellia frereana (BF) extract formulations versus Placebo upon skin moisture. Statistical comparisons were performed comparing groups A, B and C against group D through 8 weeks. Only 3% BF formulation showed significant effect;

Table 5. Skin Texture Analysis. Images were taken after 2, 4 and 8 weeks of product use of each formulation (1%, 2%, and 3% Boswellia frereana (BF) extract formulation and placebo) and were compared with baseline images;

Table 6. Statistical Significance of effect of Boswellia frereana (BF) extract formulations versus Placebo upon skin texture. Statistical comparisons were performed comparing groups A, B and C against group D through 8 weeks. Only 1% BF formulation showed significant effect (p<0.05) compared with baseline; all other improvements were insignificant;

Table 7. Fine Lines/Wrinkles Analysis. Images were taken after 2, 4 and 8 weeks of product use of each formulation (1%, 2%, and 3% Boswellia frereana (BF) extract formulation and placebo) and were compared with baseline images. All of the formulations showed statistically significant (p<0.001) improvement compared with baseline;

Table 8. Statistical Significance of effect of Boswellia frereana (BF) formulations versus Placebo upon fine lines/wrinkles. Statistical comparisons were performed comparing groups A, B and C against group D through 8 weeks. Only 3% BF formulation showed significant effect;

Table 9. Skin Clarity Analysis. Images were taken after 2, 4 and 8 weeks of product use of each formulation (1%, 2%, and 3% Boswellia frereana (BF) extract formulation and placebo) and were compared with baseline images;

Table 10. Statistical Significance of effect of Boswellia frereana (BF) extract formulations versus Placebo upon skin clarity. Statistical comparisons were performed comparing groups A, B and C against group D through 8 weeks. Only 1% BF formulation showed significant effect;

Table 11. Skin Radiance/Luminosity Analysis. Images were taken after 2, 4 and 8 weeks of product use of each formulation and were compared with baseline images. Formulations 3% BF, 1% BF and Placebo showed statistically significant improvements at week 4 and week 8 (p<0.05). Formulation 2% BF showed statistically significant improvement at week 8 (p<0.05).

### DETAILED DESCRIPTION

Materials and Methods

Double-blind Placebo-Controlled Randomized Clinical Study of Boswellia frereana extract applied as a topical application at various concentrations of Boswellia frereana (BF) extract when compared to placebo.

1.0 Trial Design

The study designed was an 8-week double blind placebo controlled study, in which one of four test materials: formulation with 1%, 2%, or 3% solution of Boswellia Frereana (BF) extract topical formulation or a placebo product used by the test panellists (81 women aged between 35-65). ˜20 subjects for placebo and each of 1, 2 and 3% formulation groups.

Placebo: Purified water, jojoba seed oil, apricot oil, squalane, laureth-7, C13-14 Isoparaffin, polyacrylamide, phenoxyethanol, ethylhexylglycerin.

For Boswellia frereana (BF) Extract Formulations, Boswellia frereana was extracted with methyl isopropanol solubilized in jojoba oil and added to the placebo base cream at a concentration of 1, 2 or 3%.

2.0 Clinical Objectives

Evaluations done at baseline, week 2, 4 and 8 to evaluate changes in skin texture; fine lines/wrinkles; skin firmness/elasticity; skin clarity (decreases the appearance of red/inflamed, brown, blotchy skin); skin radiance/luminosity; skin hydration.

Instrumental Evaluation: Cutometer for skin firmness/elasticity, Corneometer for skin hydration, Digital photography (Visia CR 2.0) to analyze changes in skin texture; fine lines/wrinkles; skin clarity (decreases the appearance of red/inflamed, brown, blotchy skin); skin radiance/luminosity.

3.0 Candidate Baseline Readings

Subjects reported to the Testing Facility for the baseline visit. A trained technician visually evaluated skin texture, global fine lines/wrinkles, skin clarity and skin radiance/luminosity on the face of each subject to determine qualification. Digital photographs were taken of the face of each subject using the Visia CR® 2.0 (Canfield Scientific, Fairfield, N.J.) and were analysed to determine changes (if any) in skin texture, global fine lines/wrinkles, skin clarity and skin radiance/luminosity. A Cutometer® measurement was taken on the face of each subject to measure skin firmness/elasticity and a Corneometer® measurement was taken to measure skin hydration. Additionally, an irritation evaluation was conducted for safety purposes. Subjects were given the test product, use instructions and a daily diary.

Formulations were applied twice daily to entire face.

4.0 Test Readings

Subjects were instructed to report to the Testing Facility after 2, 4 and 8 weeks of product use for additional irritation evaluations and photographs. Additionally, at the 2-, 4- and 8-week visits, subjects were required to complete a questionnaire.

4.1 Evaluation of Skin Texture/Roughness

At the baseline visit only, to determine qualification, a trained technician evaluated skin texture/roughness on the face of each subject according to the scale below:
0=None
1-3=Slightly rough skin texture
4-6=Moderately rough skin texture

4.2 Evaluation of Fine Lines and Wrinkles

At the baseline visit only, to determine qualification, a trained technician globally evaluated fine lines and wrinkles on the face of each subject according to the scale below:
0=None
1-3=Slight
4-6=Noticeable
7-9=Very Noticeable

4.3 Evaluation of Skin Radiance/Luminosity

At the baseline visit only, to determine qualification, a trained technician evaluated skin radiance on the face of each subject according to the scale below:
0=No dullness present/radiant complexion
1-3=Slightly dull appearance
4-6=Moderately dull appearance
7-9=Severely dull appearance

4.4 Evaluation of Skin Clarity

At the baseline visit only, to determine qualification, a trained technician evaluated skin clarity on the face of each subject according to the scale below:
0=Perfectly even complexion (no discolorations visible)
1-3=Slight areas of discolorations (dark spots, blotchiness, hyperpigmentation, etc.) visible
4-6=Moderate areas of discolorations (dark spots, blotchiness, hyperpigmentation, etc.) visible
7-9=Severe areas of discolorations (dark spots, blotchiness, hyperpigmentation, etc.) visible

4.5 Evaluation of Irritation

At each visit, a trained technician evaluated the face of each subject for irritation according to the scale below. This evaluation was for safety purposes only and was not used in determining efficacy.
0=No irritation present
+=Barely perceptible irritation present
1=Mild irritation present
2=Moderate irritation present
3=Marked irritation present
4=Severe irritation present

4.6 Evaluation of Skin Firmness—Cutometer® Measurements

At each evaluation, the firmness of the skin was measured on the face of each subject using the Cutometer®. An increase in Cutometer® measurements indicated an improvement (increase) in skin firmness. A decrease represented a worsening.

4.7 Evaluation of Skin Hydration—Corneometer® Measurements

At each evaluation, the moisture/hydration of the skin was measured on the face of each subject using the Corneometer®. An increase in Corneometer® measurements indicated an improvement (increase) in skin moisture/hydration. A decrease represented a worsening.

5.0 Digital Photography Procedure and Image Analysis

At each visit, digital images of the face of each subject were taken from the front, right and left views. In order to ensure consistency between the photographs, each subject was draped with a black cloth around the shoulders in order to eliminate the appearance of clothing in the pictures and each subject wore a black headband to pull hair off of and away from the face.

The images were analyzed using Image Pro® software (MediaCybernetics, Bethesda, Md.) to determine changes (if any) in the following parameters:
Skin texture
Global fine lines/wrinkles
Skin clarity
Skin radiance/luminosity

5.1 Skin Texture/Smoothness—Image Analysis

In order to determine changes in skin texture/smoothness, each digital image was scanned horizontally and vertically to collect the red, green and blue intensities of the pixels. The proprietary mathematical algorithm in Visia CR® uses the pixel intensities of the scanned areas to calculate the texture score based on the totals of the mean intensities of the red, green and blue pixels. Texture scores are a single number calculated based on skin features. A decrease in the texture score represented an improvement in overall skin texture.

5.2 Crow's Feet Fine Lines/Wrinkles—Image Analysis

In order to determine changes in crow's feet fine lines/wrinkles, each digital image was scanned horizontally and vertically to collect the red, green and blue intensities of the pixels. The proprietary mathematical algorithm in Visia CR® uses the pixel intensities of the scanned areas to calculate the texture score based on the totals of the mean intensities of the red, green and blue pixels. Texture scores are a single number calculated based on skin features. A decrease in the texture score represented an improvement (or decrease) in the appearance of crow's feet fine lines/wrinkles.

5.3 Skin Clarity—Image Analysis

In order to determine changes in skin clarity/blotchiness, chroma was analyzed. The degree to which a colour is free from being mixed with other colours is a good indication of its chromacity. An increase in the chroma score represented an improvement in skin clarity/blotchiness.

5.4 Skin Radiance/Luminance—Image Analysis

In order to determine any changes in skin radiance/luminance, facial luminance was analyzed. Facial luminance is a single number calculated based on the uniformity of the lightening of the image. An increase in the facial luminance score represented an improvement in overall skin luminance.

6.0 Observed Skin Improvement Analysis and Interpretation

The present study assessed effects upon skin improvement through measurement of a number of parameters that are attributed to skin health and well-being, as outlined in 4.1 to 4.7. Improvement was assessed based on a comparison of at least one, but preferably a combination of any one of said parameters, versus baseline across the entire observation period for each product versus the placebo.

7.0 Statistics

Mixed model repeat measure ANCOVA was performed on the raw analysis data readouts. Statistical comparisons were performed comparing groups A, B and C against group D across the entire 8 weeks period.

Results

Eighty-one female subjects, between the ages of 38 and 65 years, were empanelled. A total of 78 (78/81) subjects successfully completed the study. A total of 3 (3/81) test subjects were discontinued for personal reasons.

Skin Firmness/Elasticity

At baseline and after 2, 4 and 8 weeks of product use, a trained technician measured the firmness/elasticity of the skin on the face of each subject using the Cutometer®.

Objective Measures: 100% of Subjects on all formulations showed highly statistically significant improvements versus baseline at weeks 2, 4 and 8 (table 1).

All BF formulations were statistically significant over placebo through 8 weeks (table 2).

Consumer Evaluation: 90, 95, 100% (weeks 2, 4 and 8) of subjects saw improvement which were statistically significant vs placebo week 4.

Skin Moisture/Hydration

At baseline and after 2, 4 and 8 weeks of product use, a trained technician measured the moisture content of the skin on the face of each subject using the Corneometer®.

Objective Measures: 95,100, 100% of subjects on all formulations saw improvement versus baseline and mean change improvements at week 2 (44%), 4 (76%) and 8 (95%) were quantitatively greater than placebo (table 3).

3% BF formulation improvements were statistically significant over placebo through 2 weeks (table 4).

Consumer Evaluation: 95, 100, 100% (weeks 2, 4 and 8) of subjects saw an improvement which was statistically significantly different from placebo at weeks 4 and 8.

Skin Texture

At baseline and after 2, 4 and 8 weeks of product use, a trained technician took digital photographs of the face of each subject with the Visia CR® imaging system. Using ImagePro® software, the images were analyzed to determine changes in skin texture.

Objective Measures: With BF extract, 57%, 70%, and 80% of subjects saw improvement at weeks 2, 4, and 8 (table 5).

1% BF formulation improved skin texture that was statistically significant over baseline and placebo through 8 weeks (table 6).

Consumer Evaluation: 95-100% saw a statistically significant improvement in skin texture over placebo with ECM9 at weeks 2, 4, and 8 weeks.

Skin Wrinkles and Lines

At baseline and after 2, 4 and 8 weeks of product use, a trained technician took digital photographs of the face of each subject with the Visia CR® imaging system. Using ImagePro® software, the images were analyzed to determine changes in global fine lines/wrinkles.

Objective Measures: Highly statistically significant improvements were seen in 100% of subjects with 3% BF formulation vs baseline at weeks 2 and 4 (table 7) and was statistically significant over placebo through 2 weeks (table 8).

The percentage improvement observed at week 2 was more than double placebo.

Consumer Evaluation: 91-95% (weeks 2-8) of subjects saw an improvement in fine lines and wrinkles with BF formulation and was statistically different from placebo at week 2

Skin Clarity

At baseline and after 2, 4 and 8 weeks of product use, a trained technician took digital photographs of the face of each subject with the Visia CR® imaging system. Using ImagePro® software, the images were analyzed to determine changes in skin clarity.

Objective Measures: With 1% BF formulation, 52%, 52%, and 80% of subjects saw improvement at weeks 2, 4, and 8 (table 9). Improvement at week 8 was statistically significant over baseline and 3 times superior over placebo (table 10).

Consumer Evaluation: 90-95% of subjects on BF formulations saw improvements at weeks 2, 4, and 8. Week 4 and 8 improvements were statistically significant over placebo.

Skin Radiance/Luminosity

At baseline and after 2, 4 and 8 weeks of product use, a trained technician took digital photographs of the face of each subject with the Visia CR® imaging system. Using ImagePro® software, the images were analyzed to determine changes in skin radiance/luminosity.

Objective Measures: Improvements between 60%, 81% and 86% (weeks 2, 4 and 8) were seen with BF formulation over baseline (table 11) and these improvements were quantitatively greater than placebo at weeks 4 and 8.

Consumer Evaluation: 100% saw improvement in skin texture that was statistically significantly over placebo with BF Formulations at weeks 2,4 and 8 weeks.

Summary

In summary our studies have shown that a topical formulation comprising 1-3% Boswellia frereana safely, effectively and significantly improves properties of the skin with noticeable effects upon skin texture, wrinkles, firmness/elasticity, clarity, radiance, and hydration. This therefore represents an effective anti-aging compound protecting the skin from the damaging effects of inflammation, UV light and aging.

In particular a topical formulation comprising 1% Boswellia frereana was surprisingly effective at promoting improvements in texture, clarity and firmness and elasticity whereas a topical formulation comprising the higher amount of 3% Boswellia frereana was effective at promoting improvements in firmness and elasticity, moisture and combatting lines & wrinkles.

TABLE 1
Skin Firmness

| | | | |
|---|---|---|---|
| Mean | | Mean | % of Subjects with |
| Score ± | | % Change | Improvement from |
| S.D. | p-value | from Baseline | Baseline |

3% BF Formulation

| | | | | |
|---|---|---|---|---|
| Baseline | 0.403 ± 0.106 | — | — | — |
| Week 2 | 0.469* ± 0.087 | <0.001 | 19.4% | 100% |
| Week 4 | 0.528* ± 0.077 | <0.001 | 35.8% | 100% |
| Week 8 | 0.550* ± 0.061 | <0.001 | 42.8% | 100% |

2% BF Formulation

| | | | | |
|---|---|---|---|---|
| Baseline | 0.396 ± 0.108 | — | — | — |
| Week 2 | 0.452* ± 0.100 | <0.001 | 16.8% | 89% |
| Week 4 | 0.517* ± 0.097 | <0.001 | 35.0% | 100% |
| Week 8 | 0.570* ± 0.080 | <0.001 | 51.0% | 100% |

1% BF Formulation

| | | | | |
|---|---|---|---|---|
| Baseline | 0.454 ± 0.095 | — | — | — |
| Week 2 | 0.509* ± 0.093 | <0.001 | 13.9% | 75% |
| Week 4 | 0.557* ± 0.077 | <0.001 | 26.0% | 95% |
| Week 8 | 0.601* ± 0.071 | <0.001 | 36.5% | 100% |

Placebo

| | | | | |
|---|---|---|---|---|
| Baseline | 0.415 ± 0.100 | — | — | — |
| Week 2 | 0.482* ± 0.080 | <0.001 | 19.7% | 78% |
| Week 4 | 0.521* ± 0.080 | <0.001 | 29.7% | 100% |
| Week 8 | 0.554* ± 0.080 | <0.001 | 38.9% | 100% |

*Statistically significant difference from baseline, p < 0.05

TABLE 2
Statistical Comparison Versus Placebo-Skin Firmness
A vs D. P = 0.036 through 8 weeks
B vs D. P = 0.004 through 8 weeks
C vs D. P = 0.023 through 8 weeks
A-3% boswellia extract (BF) formulation
B-2% BF formulation
C-1% BF formulation
D-control base cream without BF extract

TABLE 3
Skin Hydration

| | | | |
|---|---|---|---|
| | | Mean | % of Subjects with |
| Mean Score ± | | % Change | Improvement from |
| S.D. | p-value | from Baseline | Baseline |

3% BF Formulation

| | | | | |
|---|---|---|---|---|
| Baseline | 30.8 ± 6.4 | — | — | |
| Week 2 | 43.0* ± 9.7 | <0.001 | 43.7% | 90% |
| Week 4 | 52.3* ± 11.8 | <0.001 | 76.0% | 100% |
| Week 8 | 58.0* ± 9.6 | <0.001 | 95.2% | 100% |

2% BF Formulation

| | | | | |
|---|---|---|---|---|
| Baseline | 32.6 ± 7.5 | — | — | — |
| Week 2 | 41.7* ± 9.5 | <0.001 | 30.2% | 95% |
| Week 4 | 53.4* ± 9.7 | <0.001 | 69.6% | 100% |
| Week 8 | 58.7* ± 7.7 | <0.001 | 88.1% | 100% |

1% BF Formulation

| | | | | |
|---|---|---|---|---|
| Baseline | 32.6 ± 6.7 | — | — | — |
| Week 2 | 42.2* ± 9.4 | <0.001 | 32.6% | 95% |
| Week 4 | 53.7* ± 11.5 | <0.001 | 68.9% | 100% |
| Week 8 | 59.3* ± 10.8 | <0.001 | 87.9% | 100% |

Placebo

| | | | | |
|---|---|---|---|---|
| Baseline | 31.0 ± 5.8 | — | — | — |
| Week 2 | 40.0* ± 9.0 | <0.001 | 30.7% | 100% |
| Week 4 | 52.5* ± 11.1 | <0.001 | 73.3% | 100% |
| Week 8 | 58.6* ± 9.8 | <0.001 | 92.5% | 100% |

*Statistically significant difference from baseline, p < 0.05

TABLE 4
Statistical Comparison Versus Placebo-Skin Hydration
A vs D. P = 0.023 through 2 weeks
A-3% boswellia extract (BF) formulation
B-2% BF formulation
C-1% BF formulation
D-control base cream without BF extract

TABLE 5
Skin Texture

| | | | |
|---|---|---|---|
| Mean | | Mean | % of Subjects with |
| Score ± | | % Change | Improvement from |
| S.D. | p-value | from Baseline | Baseline |

3% BF Formulation

| | | | | |
|---|---|---|---|---|
| Baseline | 193.9 ± 20.0 | — | — | — |
| Week 2 | 197.3 ± 26.3 | 0.700 | 1.8% | 43% |
| Week 4 | 192.9 ± 25.3 | 0.725 | −0.3% | 57% |
| Week 8 | 189.0 ± 29.4 | 0.092 | −2.7% | 71% |

2% BF Formulation

| | | | | |
|---|---|---|---|---|
| Baseline | 196.3 ± 19.7 | — | — | — |
| Week 2 | 194.7 ± 23.8 | 0.475 | −0.7% | 55% |
| Week 4 | 189.1 ± 16.3 | 0.113 | −3.1% | 63% |
| Week 8 | 190.4 ± 20.9 | 0.332 | −2.7% | 53% |

1% BF Formulation

| | | | | |
|---|---|---|---|---|
| Baseline | 205.1 ± 31.0 | — | — | — |
| Week 2 | 202.6 ± 35.8 | 0.579 | −1.0% | 57% |
| Week 4 | 198.9 ± 32.1 | 0.105 | −3.2% | 70% |
| Week 8 | 194.9* ± 31.4 | 0.002 | −5.3% | 80% |

Placebo

| | | | | |
|---|---|---|---|---|
| Baseline | 210.4 ± 23.3 | — | — | — |
| Week 2 | 208.5 ± 26.0 | 0.966 | −0.3% | 50% |
| Week 4 | 198.7 ± 20.4 | 0.090 | −4.7% | 61% |
| Week 8 | 199.9 ± 23.1 | 0.060 | −4.5% | 72% |

*Statistically significant difference from baseline, p < 0.05

TABLE 6
Statistical Comparison Versus Placebo-Skin Texture
C vs D. P = 0.048 through 8 weeks
A-3% boswellia extract (BF) formulation
B-2% BF formulation
C-1% BF formulation
D-control base cream without BF extract

TABLE 7
Fine Lines/Wrinkles

| | | | |
|---|---|---|---|
| Mean | | Mean | % of Subjects with |
| Score ± | | % Change | Improvement from |
| S.D. | p-value | from Baseline | Baseline |

3% BF Formulation

| | | | | |
|---|---|---|---|---|
| Baseline | 1019.0± | — | — | |
| Week 2 | 823.9* ± 103.6 | <0.001 | −17.5% | 100% |
| Week 4 | 774.0* ± 85.3 | <0.001 | −22.3% | 100% |
| Week 8 | 787.7* ± 126.8 | <0.001 | −21.2% | 95% |

2% BF Formulation

| | | | | |
|---|---|---|---|---|
| Baseline | 1017.7 ± | — | — | — |
| Week 2 | 881.2* ± | <0.001 | −11.7% | 100% |
| Week 4 | 875.9* ± | <0.001 | −13.6% | 95% |
| Week 8 | 803.9* ± | <0.001 | −20.3% | 100% |

1% BF Formulation

| | | | | |
|---|---|---|---|---|
| Baseline | 1016.3± | — | — | — |
| Week 2 | 936.2*± | <0.001 | −6.3% | 86% |
| Week 4 | 840.9* ± 92.3 | <0.001 | −16.6% | 100% |
| Week 8 | 801.7*± | <0.001 | −20.8% | 100% |

Placebo

| | | | | |
|---|---|---|---|---|
| Baseline | 984.3 ± 133.7 | — | — | — |
| Week 2 | 899.4*± | <0.001 | −8.4% | 94% |
| Week 4 | 827.9*± | <0.001 | −15.5% | 100% |
| Week 8 | 776.3*± | <0.001 | −20.7% | 100% |

*Statistically significant difference from baseline, p < 0.05

TABLE 8
Statistical Comparison Versus Placebo-Lines/Wrinkles
A vs D. P = 0.039 through 2 weeks
A-3% boswellia extract (BF) formulation
B-2% BF formulation
C-1% BF formulation
D-control base cream without BF extract

TABLE 9
Skin Clarity

| | | | |
|---|---|---|---|
| Mean | | Mean | % of Subjects with |
| Score ± | | % Change | Improvement from |
| S.D. | p-value | from Baseline | Baseline |

3% BF Formulation

| | | | | |
|---|---|---|---|---|
| Baseline | 17.2 ± 2.5 | — | — | — |
| Week 2 | 17.3 ± 2.5 | 0.828 | 0.6% | 52% |
| Week 4 | 17.3 ± 2.5 | 0.960 | 0.6% | 52% |
| Week 8 | 17.0 ± 2.5 | 0.212 | −1.0% | 38% |

2% BF Formulation

| | | | | |
|---|---|---|---|---|
| Baseline | 17.7 ± 2.1 | — | — | — |
| Week 2 | 17.4 ± 2.4 | 0.294 | −1.7% | 40% |
| Week 4 | 17.4* ± 2.2 | 0.651 | −0.8% | 42% |
| Week 8 | 17.5* ± 2.4 | 0.798 | −0.6% | 53% |

1% BF Formulation

| | | | | |
|---|---|---|---|---|
| Baseline | 17.7 ± 2.4 | — | — | — |
| Week 2 | 17.6 ± 2.7 | 0.254 | −1.3% | 38% |
| Week 4 | 17.7 ± 2.4 | 0.349 | −0.7% | 50% |
| Week 8 | 18.2* ± 2.4 | 0.017 | 1.9% | 80% |

Placebo

| | | | | |
|---|---|---|---|---|
| Baseline | 18.6 ± 2.8 | — | — | — |
| Week 2 | 18.1 ± 2.2 | 0.027 | −1.9% | 17% |
| Week 4 | 18.1 ± 2.3 | 0.108 | −2.2% | 39% |
| Week 8 | 18.1 ± 2.0 | 0.154 | −1.7% | 28% |

*Statistically significant difference from baseline, p < 0.05

TABLE 10
Statistical Comparison Versus Placebo-Clarity
C vs D. P = 0.002 through 8 weeks
A-3% boswellia extract (BF) formulation
B-2% BF formulation
C-1% BF formulation
D-control base cream without BF extract

TABLE 11
Skin Radiance/Luminosity

| | | | |
|---|---|---|---|
| Mean | | Mean | % of Subjects with |
| Score ± | | % Change | Improvement from |
| S.D. | p-value | from Baseline | Baseline |

3% BF Formulation

| | | | | |
|---|---|---|---|---|
| Baseline | 138.0 ± 10.9 | — | — | |
| Week 2 | 138.0 ± 10.8 | 0.907 | 0.2% | 43% |
| Week 4 | 140.3* ± 11.1 | 0.005 | 1.9% | 81% |
| Week 8 | 141.7* ± 11.2 | <0.001 | 2.9% | 86% |

2% BF Formulation

| | | | | |
|---|---|---|---|---|
| Baseline | 138.7 ± 7.3 | — | — | — |
| Week 2 | 139.0 ± 8.6 | 0.784 | 0.2% | 60% |
| Week 4 | 140.5 ± 7.7 | 0.145 | 1.3% | 53% |
| Week 8 | 142.3* ± 9.1 | 0.003 | 2.5% | 79% |

1% BF Formulation

| | | | | |
|---|---|---|---|---|
| Baseline | 139.7 ± 8.7 | — | — | — |
| Week 2 | 140.4 ± 9.1 | 0.448 | 0.6% | 52% |
| Week 4 | 141.5 ± 9.0 | 0.024 | 1.7% | 80% |
| Week 8 | 141.6* ± 9.0 | 0.027 | 1.8% | 65% |

Placebo

| | | | | |
|---|---|---|---|---|
| Baseline | 135.0 ± 11.5 | — | — | — |
| Week 2 | 136.1 ± 10.5 | 0.212 | 0.9% | 61% |
| Week 4 | 139.1* ± 9.2 | 0.002 | 3.2% | 78% |
| Week 8 | 139.9* ± 8.6 | 0.005 | 3.9% | 72% |

*Statistically significant difference from baseline, p < 0.05

1. A composition formulated for topical application for use in skin improvement or repair comprising an extract of Boswellia frereana, wherein said extract is present in an amount at or between 1-3% by weight of the formulation.

2. The composition according to claim 1 comprising a mixture of components which are present in the oleo-resin of Boswellia frereana and which are soluble in an organic solvent.

3. The composition according to claim 2 comprising a mixture of components which are soluble in a polar solvent.

4. The composition according to claim 3 wherein said solvent is selected from the group comprising: methanol, ethanol, isoproponal, methyl isopropanol and derivatives thereof.

5. The composition according to claim 4 wherein said solvent is methyl isopropanol.

6. The composition according to claim 1 wherein said extract is the essential oil of Boswellia frereana.

7. The composition according to claim 1 wherein said extract does not contain Boswellic acid.

8. The composition according to claim 1 wherein said extract is present in an amount selected from the group comprising: 1%, 2%, 3% and every 0.1% integer there between.

9. The composition according to claim 1 wherein said extract is present in an amount selected from the group consisting of: 1%, 2%, 3% and every 0.1% integer there between.

10. The composition according to claim 1 wherein said extract comprises the pentacyclic triterpene lupeol and/or epi-lupeol or a derivative or a salt thereof

11. The composition according to claim 1 wherein said extract additionally or alternatively comprises β-amyrin, α-amyrin, α-phellandrene dimers, α-thujene and α-phellandrene or their isomers and salts thereof.

12. The composition according to claim 1 further comprising at least one carrier or excipient.

13. The composition according to claim 12 wherein said carrier or excipient is a pharmaceutical or veterinary carrier or excipient.

14. The composition according to claim 1 further comprising a moisturiser or emollient.

15. The composition according to claim 1 wherein said composition is formulated as a cream, serum, ointment, jelly, solution or suspension.

16. The composition according to claim 15 wherein said composition is formulated as a cream.

17. (canceled)

18. A method for skin improvement or repair comprising applying to the surface of the skin of an individual an effective amount of a topical formulation comprising an extract of Boswellia frereana, wherein the extract is present in an amount at or between 1-3% by weight of the formulation.

19. The method according to claim 18 wherein said composition is applied at least twice daily to the surface of the skin.

20. The method according to claim 18 wherein said method is cosmetic.

21. A composition formulated for topical application for use in skin improvement or repair comprising:
an extract of Boswellia frereana; and
a pharmaceutically or veterinary acceptable carrier or excipient;
wherein said extract is present in an amount at or between 1-3% by weight of the formulation.
